(19) United States
(12) Patent Application Publication
(10) Pub. No.: US 2020/0069760 A1
(43) Pub. Date: Mar. 5, 2020

(54) PERCYTE COMPOSITION

(71) Applicant: DuraScience Inc., New York, NY (US)

(72) Inventor: James Nitit Mah, New York, NY (US)

(73) Assignee: DuraScience Inc., New York, NY (US)

(21) Appl. No.: 16/679,281

(22) Filed: Nov. 10, 2019

(51) Int. Cl. A61K 36/78; A61K 36/07; A61K 36/424; A23L 33/105

(52) U.S. Cl. CPC A61K 36/78; A61K 36/07; A23V 2002/00; A23L 33/105; A61K 36/424

(57) ABSTRACT

In an embodiment, a nutritional supplement composition is provided. The nutritional supplement composition is provided including a Houttuynia cordata extract of the Houttuynia cordata strain, maitake extract of the Grifola frondosa strain of mushrooms and Gynostemma extract of the leaf of the Gynostemma pentaphyllum strain. The nutritional supplement is in the form of a compound known as PERCYTE™

### BACKGROUND

In the past, we commonly used all parts of natural foods when we consumed a meal. Human eating patterns naturally accessed nutrients available from what was eaten and very little nutrients were wasted as a result. For example, when eating fish, the muscular parts of the fish were consumed along with other parts such as connective tissue. When eating plants, the edible portion of the plant was consumed in its entirety. Our bodies would then filter out what was not consumable, such as the outer shell of a kernel of corn for example. As this occurred, our bodies would extract what nutrients could be found in the food to sustain and grow the human form. This allowed use of local foods to provide nutrients which were available, but was somewhat limited in that foods not available locally could not be used in most cases. Over time, we learned to travel and to ship food from where it was produced to where it could be consumed. Additionally, we learned to prepare foods, removing portions of the food which were either inedible, or undesirable. Removing the inedible parts typically provided some benefit, while parts that were simply undesirable sometimes held valuable ingredients.

Houttuynia cordata is a flowering plant of southeast asia. The Grifola frondosa strain of mushrooms grows wild in China, Japan and North America. Gynostemma extract comes from the leaf of the Gynostemma pentaphyllum strain. The Gynostemma pentaphyllum strain is known to grow in the mountainous regions of southern China and northern Vietnam.

### DETAILED DESCRIPTION

A system, method and apparatus is provided for a PERCYTE™ compound. The specific embodiments described in this document represent exemplary instances of the present invention, and are illustrative in nature rather than restrictive.

In the following description, for purposes of explanation, numerous specific details are set forth in order to provide a thorough understanding of the invention. It will be apparent, however, to one skilled in the art that the invention can be practiced without these specific details. In other instances, structures and devices are shown in block diagram form in order to avoid obscuring the invention.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments.

Houttuynia cordata has been shown to assist in combatting oral infectious diseases. Houttuynia cordata includes polyphenolic flavonoids, such as quercetin, quercitrin, hyperoside, and rutin. All of these exhibit anti-inflammatory, antioxidant, and antibacterial properties. Quercitrin has also been shown to decrease damage from UVB rays.

Maitake extract has been shown to potentially lower blood sugar levels. It has also been shown to reduce the effects of polycystic ovary syndrome. Maitake extract also tends to lower blood pressure. Moreover, maitake extract potentially slows growth of cancerous tumors. Also, maitake extract potentially boosts the immune system

Gynostemma pentaphyllum is known for activating AMPK, a molecule that acts as a enhances production of ATP. AMPK responds to low ATP levels. When AMPK is activated, it turns up processes in cells and mitochondria that enhance conversion of food into ATP. This helps restore ATP levels. ATP is adenosine triphosphate, and is the preferred source of energy in cells. When a cell runs out of ATP, it uses the product of energy production from ATP, namely ADP or adenosine diphosphate. However, use of ADP to produce energy is less efficient and produces more residual effects such as cramping or soreness.

Restoring ATP is therefore important. In addition to restoring ATP, AMPK appears to adjust gene expression in ways that result in increased capacity to make more cellular energy in the future.

Gynostemma pentaphyllum promotes antioxidant defenses, enhances insulin signaling, and acts as neuroprotective support. Also, feeding Gynostemma pentaphyllum to animals has been shown to shift the gut microbiota in ways that have been associated with better shape and weight, along the lines of a probiotic. This potentially produces a healthier metabolism.

Gynostemma pentaphyllum is also used for high blood pressure, improving heart function and high cholesterol. It is also used for increasing stamina and endurance, strengthening the immune system, increasing resistance to environmental stress, preventing hair loss and improving memory.

The benefit of the composition includes restoring and enhancing cells. Our cells constantly deteriorate in daily life. If there is no support to restore the deterioration, cellular damage will affect the body functions. PERCYTE™ composition is a supplementary factor that can restore the cells to the body balance without drug and substance usage. When cells are restored, in the next process, PERCYTE™ composition may accordingly boost cell strength, equivalent to increasing the effectiveness and the number of cells, while maximizing physical and mental abilities After the cellular restoration and boosting the cell strength, the next challenge is the protection to maintain the long-lasting quality of the cells. PERCYTE™ dietary supplement may help to protect and delay the cellular deterioration. This will also result in improving the immune system, as the immune system no longer needs to activate against inflammation or other forms of deterioration. When the cell functions are systematically restored, boosted and protected, PERCYTE™ dietary supplement may directly enhance physical and mental capacity to go beyond limits at each age and to live life fully as the pace of life requires a strong response to maintain youth and vigor.

The health benefits of PERCYTE™ are also potentially of value to children, and help to develop the strength of new cells and protect cells from early deterioration. Both are beneficial to the development of body, intelligence, memory, and positive emotions. The physical results may include i) restore cells for normal growth in each stage, ii) recover from illness, iii) build muscle, v) boost the immune system, vi) reduce the risk of the incidence of disease, vii) promote growth to maximum effectiveness, viii) adjust height, ix) improve body growth, x) efficient immunity and xi) increase energy. The wound and injury healing properties are potentially of particular assistance to children, as the nutrients provided assist specifically with structures often injured in sports or other childhood activities.

The composition also helps to restore the old damaged cells, repair damaged body tissues, delay the cellular deterioration, inhibit free radicals, and help the process of skin cell renewal in adults for glowing skin and younger appearance. Providing building blocks as previously mentioned contribute to enhancing balance, reducing exhaustion from stress and demands of healing, improving concentration and work efficiency, reducing healing time and longer term pain, and boosting the immune system to stay healthy

Physical results may also include: a radiant and vibrant skin, younger appearance; reducing fatigue at work; restoring the reproductive system; refreshing the body; boosting the immune system; protecting against free radical formation; and preventing premature aging.

The compounds are typically dried and ground or milled to small particles, and subsequently combined in the desired ratio. The combination may remain as a powder to be added to food or pressed in a tablet. Alternatively, the compounds may be mixed in an aqueous or other liquid solution and thereby provided in liquid form.

In an embodiment, a preferred dosage, the total compound blended is 225 mg, pressed into a single tablet. The compound may be sold under the PERCYTE™ name. The dosage may include a combination of Houttuynia cordata extract in the amount of 100 mg, maitake extract in the amount of 75 mg and Gynostemma extract in the amount of 50 mg. However, other ratios of the ingredients may be provided, and smaller or larger dosages may be used for particular applications.

Study of PERCYTE™ Effectiveness

A study of users of a trial formulation was conducted. The study focused in part on healing and immunology factors, but also considered other possible results. Participants reported the following results:

Ninety-four percent (94%) of participants reported reduced time to heal wounds, with a typical response of a sixty-five percent (90%) shorter time to heal as a measured result.

Eighty-three percent (83%) of participants reported increased endurance during exercise, with a typical measured result of fifty percent (50%) greater endurance.

Eighty-five percent (85%) of participants reported better sleep quality. The participants reported a measured result of eighty percent (80%) better sleep quality prior to use of PERCYTE™.

Ninety-four percent (94%) of participants reported better resistance to upper respiratory tract infections. The participants reported incidence of such infections decreasing to one fifth of incidence prior to use of PERCYTE™.

Eighty-four percent (84%) of participants also reported decreases in lipid levels, with a measured result of typically a thirty percent (30%) reduction in lipid levels.

Eighty percent (80%) of participants reported increased muscle mass, with a typical result of a twenty percent (20%) increase in total body muscle mass.

Eighty-six percent (86%) of participants were found to have a typical ten percent (10%) improvement in blood pressure, potentially as a result of improved nitric oxide signaling.

Eighty-three percent (83%) of participants reported better hair and nail quality. The participants reported an eighty percent (80%) improvement in hair and nail quality compared to indications prior to use of PERCYTE™.

Ninety percent (90%) of participants reported increased libido, typically reporting an eighty percent (80%) increase in their perceived libido after use of PERCYTE™

Additionally, ninety percent (90%) of participants reported improved mood, typically measuring as a sixty percent (60%) increase (improvement) in measured depression score.

One skilled in the art will appreciate that although specific examples and embodiments of the system and methods have been described for purposes of illustration, various modifications can be made without deviating from present invention. For example, embodiments of the present invention may be applied to many different types of databases, systems and application programs. Moreover, features of one embodiment may be incorporated into other embodiments, even where those features are not described together in a single embodiment within the present document.

What is claimed is:

1. A nutritional composition consisting of:
Houttuynia cordata extract of the Houttuynia cordata strain;
maitake extract of the Grifola frondosa strain of mushrooms; and
Gynostemma extract of the leaf of the Gynostemma pentaphyllum strain.

2. The composition of claim 1, wherein:
Houttuynia cordata extract in the amount of 100 mg;
maitake extract in the amount of 75 mg; and
Gynostemma extract in the amount of 50 mg.
